(19) United States
(12) Patent Application Publication
(10) Pub. No.: US 2022/0395163 A1
(43) Pub. Date: Dec. 15, 2022

(54) ROTATION ASSEMBLY FOR ENDOSCOPE LENS

(71) Applicant: Covidien LP, Mansfield, MA (US)

(72) Inventors: Hongwen Yao, Shanghai (CN); Yong Guo, Shanghai (CN)

(21) Appl. No.: 17/776,708

(22) PCT Filed: Nov. 15, 2019
(86) PCT No.: PCT/CN2019/118706

§ 371 Date: May 13, 2022

(51) Int. Cl. A61B 1/00; A61B 1/05

(52) U.S. Cl. CPC A61B 1/00183; A61B 1/05; A61B 1/00096; A61B 1/00066

(57) ABSTRACT

An endoscope includes a handle assembly, an elongate shaft extending distally from the handle assembly, a tip disposed at a distal end of the elongate shaft, an imaging device disposed within the tip, and a rotation assembly operably coupled to the imaging device for rotating the imaging device within the tip.

### CROSS-REFERENCE TO RELATED APPLICATION

This application is a National Stage Application under 35 U.S.C. § 371(a) of PCT/CN2019/118706, filed Nov. 15, 2019. The entire contents of the foregoing application is incorporated by reference herein.

### FIELD

The present technology is generally related to medical devices and, more particularly, to endoscopes including a rotation assembly for rotating the endoscope lens.

### BACKGROUND

Endoscopes are introduced through an incision or a natural body orifice to observe internal features of a body. Conventional endoscopes include a light transmission pathway, including a fiber light guide, for transmitting light from an external light source through the endoscope to illuminate the internal features of the body. Recently, endoscopes include an internal light source, instead of the combination of the external light source and the fiber light guide, to directly illuminate the internal features of the body. Conventional endoscopes also include an image retrieval pathway for transmitting images of these internal features back to an eyepiece or external video system for processing and display on an external monitor.

During minimally invasive medical procedures, endoscopy provides physicians with visualization of the internal features of the body for accurate diagnosis and/or treatment. For example, rigid endoscopy is utilized in common surgical procedures, such as appendectomy, stomach surgeries, duodenal ulcer perforation repair, hernia repair, colectomy, splenectomy, adrenalectomy, ovarian cyst removal, ectopic pregnancy, hysterectomy, and so on.

In use, an endoscope may need to be repositioned or rotated to increase or change the field of view. When the camera is fixedly mounted in the endoscope, rotation of the endoscope will also rotate the image transmitted back to the eyepiece or the external monitor. When components of the camera are rotatable, the endoscope typically includes complicated optical and/or mechanical designs to maintain imaging focus during rotation.

### SUMMARY

The disclosure relates generally to an endoscope including a rotation assembly that rotates the whole imaging device (e.g., camera module) of the endoscope. The lens of the endoscope is a single element that is short in total track length and maintains good image quality during rotation.

In an aspect, the disclosure provides an endoscope including a handle assembly, an elongate shaft extending distally from the handle assembly along a central longitudinal axis, a tip disposed at a distal end of the elongate shaft, an imaging device disposed within the tip, and a rotation assembly operably coupled to the imaging device for rotating the imaging device within the tip. The rotation assembly extends from the tip, through the elongate shaft, and into the handle assembly.

The tip may include a distal surface extending along an axis that is angled with respect to the central longitudinal axis of the elongate shaft. The imaging device may include a lens extending through the tip along a longitudinal axis that is perpendicular to the axis defined by the distal surface of the tip.

The imaging device may be disposed within a cavity defined in the tip, and a transparent imaging cover may be disposed over the imaging device. The transparent imaging cover may be positioned within an imaging window defined in the distal surface of the tip.

The rotation assembly may include a lens bearing that supports the lens within the tip. The lens bearing may include an outer ring coupled to the tip and an inner ring coupled to the lens, the inner ring rotatable relative to the outer ring such that the lens is rotatable within the tip.

The rotation assembly may include a lens plate secured to the lens of the imaging device, and a rotation shaft operably coupled to the lens plate, wherein rotation of the rotation shaft rotates the lens plate and the lens. The rotation assembly may include a cross shaft interconnecting the lens plate and the rotation shaft such that the lens plate and the rotation shaft are pivotably coupled to one another about two axes defined by the cross shaft.

The rotation shaft may extend from the lens plate proximally through the elongate shaft and into the handle assembly. The rotation shaft may be radially rotatable around the central longitudinal axis. The rotation assembly may include a rotation shaft bearing supporting the rotation shaft in the handle assembly.

The rotation assembly may include a magnet base disposed within the handle assembly and radially rotatable therein around the central longitudinal axis. The magnet base may include at least one magnet, and the rotation shaft may extend through the magnet base in fixed relation relative thereto. The rotation assembly may include a rotating ring disposed on the handle assembly and may be rotatable relative to the handle assembly. The rotating ring may include at least one magnet having a polarity opposite the polarity of the at least one magnet of the magnet base such that rotation of the rotating ring causes a corresponding rotation of the magnet base and the rotation shaft.

The at least one magnet of the magnet base may be a plurality of magnets disposed radially around the magnet base in spaced relation relative to each other, and the at least one magnet of the rotating ring may be a plurality of magnets disposed radially around the rotating ring in spaced relation relative to each other. Each magnet of the plurality of magnets of the magnet base may be paired with one of the plurality of magnets of the rotating ring, the paired magnets having opposite polarities. The plurality of magnets of the magnet base may alternate in polarity around the magnet base, and the plurality of magnets of the rotating ring may alternate in polarity around the rotating ring.

The handle assembly may include a proximal housing formed from first and second housing halves that cooperate to define a grip portion and a mount portion. The rotating ring may be supported on the mount portion of the proximal housing. The rotation assembly may include a rotating ring bearing including an outer ring coupled to the rotating ring and an inner ring coupled to the mount portion of the proximal housing, the outer ring being rotatable relative to the inner ring such that the rotating ring is rotatable relative to the proximal housing.

The handle assembly may include a distal housing, and the second housing half of the proximal housing may extend distally beyond the first housing half to support the distal housing thereon. The distal housing may define a central opening therethrough in which the elongate shaft is supported and connected to the handle assembly.

In another aspect, the disclosure provides a method of changing the field of view of an endoscope, the method including rotating a rotation assembly of an endoscope, the rotation assembly operably coupled to an imaging device disposed within a tip of the endoscope to change the orientation of the imaging device relative to the tip, the rotation assembly extending from the tip, through an elongated shaft of the endoscope, and into a handle assembly of the endoscope.

Rotating the rotation assembly of the endoscope may include applying torque to a rotating ring of the rotation assembly, the rotating ring disposed on the handle assembly of the endoscope.

The method may further include rotating the endoscope in its entirety to cause a corresponding rotation of the elongate shaft and the tip, the tip including a distal surface that extends along an axis that is angled with respect to a central longitudinal axis of the elongate shaft.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a side view of an endoscope system including an endoscope in accordance with an embodiment of the disclosure;

FIG. 2 is a cross-sectional view of the endoscope of FIG. 1;

FIG. 3 is an enlarged cross-sectional view of a distal end portion of the endoscope of FIG. 2;

FIGS. 4 and 5 are perspective views of the distal end portion of the endoscope of FIG. 3, with an elongate shaft of the endoscope removed therefrom;

FIG. 6 is an enlarged cross-sectional view of a handle assembly of the endoscope of FIG. 2; and

FIG. 7 is an end cross-sectional view of the handle assembly of FIG. 6, taken along section line 7-7 of FIG. 6.

### DETAILED DESCRIPTION

Embodiments of the disclosure are now described in detail with reference to the drawings in which like reference numerals designate identical or corresponding elements in each of the several views. Throughout this description, the term “proximal” refers to a portion of a system, a device, or a component thereof, that is closer to a user, and the term “distal” refers to a portion of the system, the device, or the component thereof, that is farther from the user.

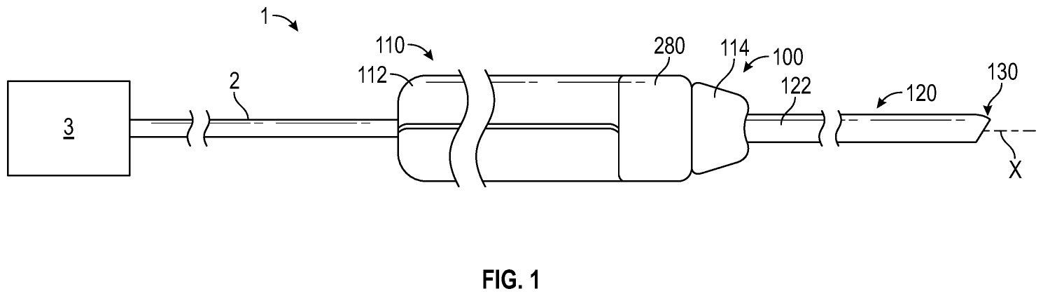

Referring now to FIG. 1, an endoscope 100 of the disclosure is shown as part of an endoscope system 1. The endoscope system 1 includes the endoscope 100 and a cable 2 coupled to the endoscope 100 and an external system component 3. The external system component 3 may be a system control center including a display (e.g., a monitor). The system control center may include software and hardware components for powering and/or controlling a lighting device and/or an imaging device of the endoscope, and/or for processing images captured by the imaging device and outputting video signals to the display to display the captured images. Alternatively, the endoscope 100 may include integrated system controls (e.g., a processing unit, power chip, and user controls incorporated into the endoscope) such that the external system component 3 is a display, the endoscope 100 may include an integrated display (e.g., an eyepiece or screen coupled to the endoscope) such that the external system component 3 is a system control center, or the endoscope 100 may be a self-contained device including both integrated system controls and an integrated display.

The endoscope 100 includes a handle assembly 110 and an elongate shaft or insertion tube 120 extending distally from the handle assembly 110 and terminating at a tip 130. The handle assembly 110 is used by a user to control and manipulate the endoscope 100, and may include actuating elements (not explicitly shown), such as buttons triggers, etc., for functional control of the endoscope 100.

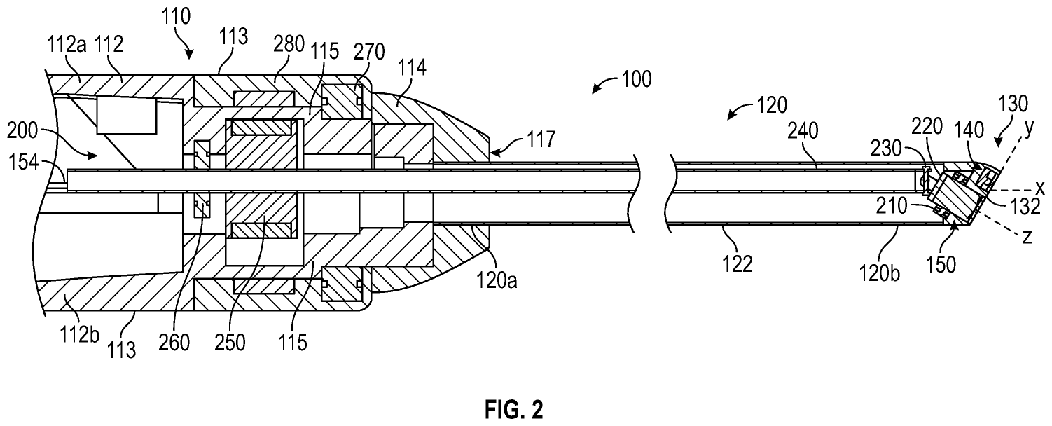

As shown in FIGS. 1 and 2, the handle assembly 110 includes a proximal or handle housing 112, a distal housing 114 and a rotating ring 280 disposed between the proximal and distal housings 112, 114. The proximal housing 112 is formed from first and second housing halves 112a, 112b that cooperate to define a grip portion 113 and a mount portion 115 depending from the grip portion 113. The grip portion 113 is configured for handling by a user, and the mount portion 115 is configured to support the distal housing 114 and/or the rotating ring 280 thereon. As seen in FIG. 2, the first and second housing halves 112a, 112b support the rotating ring 280 thereon, and the second housing half 112b extends distally beyond the first housing half 112a to support the distal housing 114 thereon. The distal housing 114 defines a central opening 117 therethrough in which the elongate shaft 120 is supported and connected to the handle assembly 110. The rotating ring 280 is rotatable relative to the proximal and distal housings 112, 114, as described in further detail below.

The elongate shaft 120 includes a rigid cylindrical wall 122 extending distally from the handle assembly 110 from a proximal end portion 120a thereof to a distal end portion 120b thereof along a central longitudinal axis “X.” The distal end portion 120b of the elongate shaft 120 terminates at the tip 130.

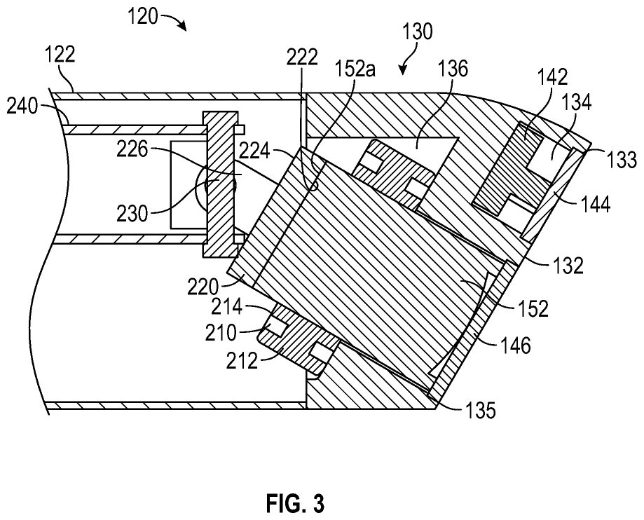

As shown in FIGS. 2 and 3, the tip 130 includes a lighting device 140 and an imaging device or camera 150 disposed therein, and a distal surface or wall 132 of the tip 130 includes a first or lighting window 133 and a second or imaging window 135 disposed over the respective lighting and imaging devices 140, 150 to provide lighting and imaging pathways of the endoscope 100. The distal surface 132 of the tip 130 extends along an axis “Y” that is angled with respect to the central longitudinal axis “X of the elongate shaft 120 such that rotation of the endoscope 100 changes the orientation of the tip 130 and thus, the field of view within a surgical site.

The lighting device 140 is disposed within a first cavity 134 defined in the tip 130 and includes a light source 142 having one or more light emitting elements (not explicitly shown), such as light-emitting diodes. A transparent lighting cover 144, such as a glass or plastic substrate, is disposed over the lighting device 140 within the lighting window 133 defined in the distal surface 132 of the tip 130 (e.g., flush with the distal surface 132 of the tip 130) to protect the lighting device 140. The transparent lighting cover 144 is disposed distal to, or in front of, the light source 142, and is isolated (e.g., separate) from the light source 142.

The imaging device 150 is disposed within a second cavity 136 defined in the tip 130 and includes a lens 152 disposed therein and an image sensor (not explicitly shown) disposed proximal of the lens 152. The lens 152 extends along a longitudinal axis “Z” that is perpendicular to the axis “Y” defined by the distal surface 132 of the tip 130. The imaging device 150 is mounted directly within the second cavity 136 of the tip 130, and does not include relay lenses and/or prisms. A transparent imaging cover 146, such as a glass or plastic substrate, is disposed over the imaging device 150 within the imaging window 135 defined in the distal surface 132 of the tip 130 (e.g., flush with the distal surface 132 of the tip 130) to protect the imaging device 150. The transparent imaging cover 146 is disposed distal to, or in front of, the lens 152, and is isolated (e.g., separate) from the lens 152.

With reference again to FIG. 2, the endoscope 100 includes a rotation assembly 200 extending from the tip 130, through the elongate shaft 120, and into the handle assembly 110 for rotating the imaging device 150 disposed within the tip 130. The rotation assembly 200 includes a lens bearing 210, a lens plate 220, a cross shaft 230, a rotation shaft 240, a magnet base or holder 250, a rotation shaft bearing 260, a rotating ring bearing 270, and the rotating ring 280.

Turning now to FIG. 3, the lens 152 is supported within the second cavity 136 of the tip 130 by the lens bearing 210. The lens bearing 210 includes an outer ring 212 coupled (e.g., fixed) to an inner surface of the tip 130 and an inner ring 214 coupled to the lens 152 (e.g., disposed around the body of the lens) and rotatable relative to the outer ring 212 such that the lens 152 is rotatable with respect to the tip 130 and the transparent imaging cover 146.

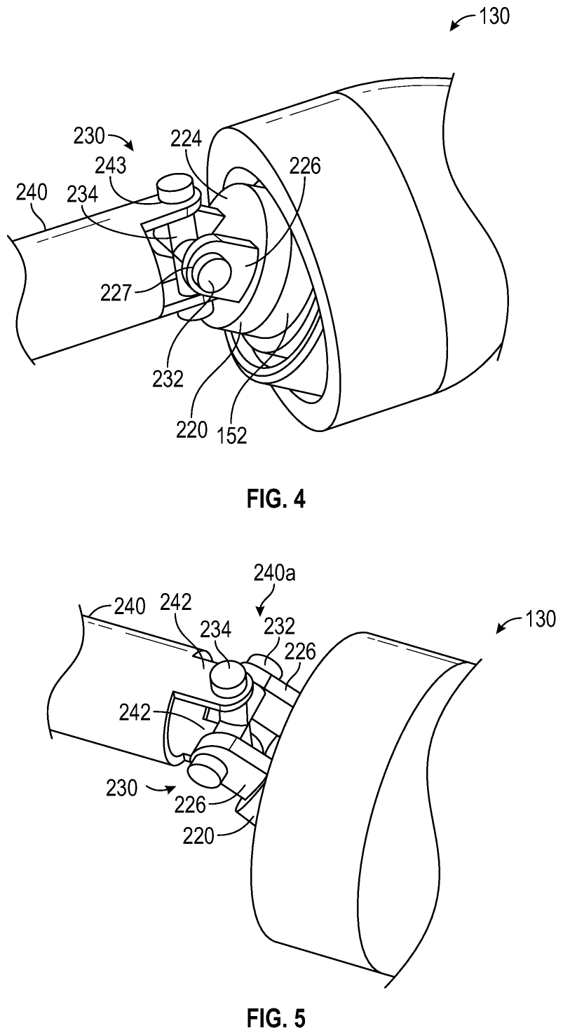

As shown in FIGS. 2-5, a lens plate 220 is coupled to the imaging device 150 and to the rotation shaft 240 by the cross shaft 230. The lens plate 220 includes a distal surface 222 fixed to a proximal end 152a of the lens 152 and a proximal surface 224 including a pair of fins 226 extending proximally therefrom. The pair of fins 226 includes opposed openings 227 through which a first rod 232 of the cross shaft 230 is pivotally secured. A distal end 240a of the rotation shaft 240 includes a pair of fins 242 including opposed openings 243 through which a second rod 234 of the cross shaft 230 is pivotally secured. It should be understood, however, that other connectors may be utilized to pivotally couple the lens plate 220 and the rotation shaft 240 to the cross shaft 230, as is within the purview of those skilled in the art. As seen in FIGS. 4 and 5, the lens plate 220, the cross shaft 230, and the rotation shaft 240 are rotatable, but are not slidable within the endoscope 100. The cross shaft 230 enables rotation of the lens plate 220 and the rotation shaft 240 about the two axes defined by the first and second rods 232, 234 of the cross shaft 230, and reliable transmission of torque and motion in the presence of an angle between the two axes.

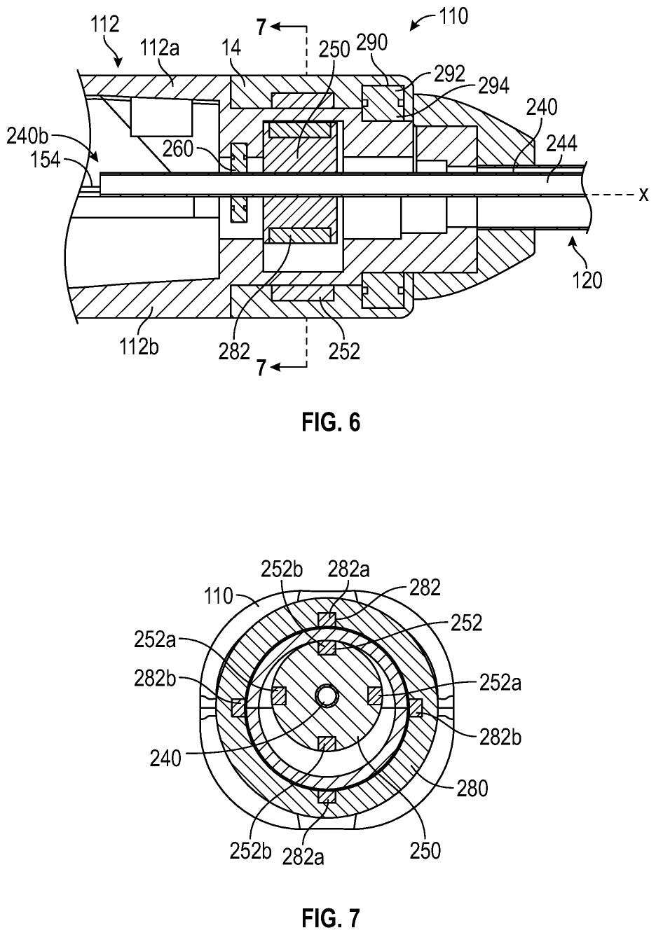

As shown in FIGS. 2 and 6, the rotation shaft 240 extends proximally from the cross shaft 230 through the elongate shaft 120 of the endoscope 100 and into the handle assembly 110 such that a proximal end 240b of the rotation shaft 240 is disposed within the proximal housing 112 of the handle assembly 110. The rotation shaft 240 extends through, and is secured within, the magnet base 250 and the rotation shaft bearing 260, which supports the rotation shaft 240 within the handle housing 110. The rotation shaft 240 includes a hollow body 244 such that wires 154 of the imaging device 150 may be passed therethrough.

As shown in FIGS. 6 and 7, the magnet base 250 includes at least one magnet 252 disposed within or affixed to the magnet base 250 (e.g., about an outer surface of the magnet base), and the rotating ring 280 includes at least one magnet 282 disposed within or affixed to the rotating ring 280 (e.g., about an inner surface of the rotating ring). The at least one magnet 252 of the magnet base 250 and the at least one magnet 282 of the rotating ring 280 have opposite polarities so that they attract one another. Accordingly, when the rotating ring 280 is rotated relative to the handle assembly 110, the at least one magnet 282 of the rotating ring 280 attracts the at least one magnet 252 of the magnet base 250 such that the magnet base 250 rotates with the rotating ring 280. As the rotation shaft 240 is fixedly or non-rotatably disposed within the magnet base 250, the rotation shaft 240 also rotates with the magnet base 250 which, as best seen in FIG. 7, rotates within the handle assembly 110 radially around the central longitudinal axis “X” (FIG. 6). The magnet base 250 and rotating ring 280 are thus not mechanically connected to each other, but utilize the magnets 252, 282 to transmit torque and link the rotation shaft 240 and the rotating ring 280 together.

As seen in FIG. 7, the magnet base 250 and the rotating ring 280 may each include a plurality of magnets 252, 282 that are disposed in radially around the respective magnet base 250 and rotating ring 280 in spaced relation relative to each other. The magnets 252, 282 are paired such that opposed magnets 252, 282 have opposite polarities. In embodiments, the plurality of magnets 252, 282 of the magnet base 250 and the rotating ring 280 alternate in polarity such that a first magnet 252a, 282a has a first polarity and a second magnet 252b, 282b has a second polarity opposite the first polarity.

With reference again to FIGS. 6 and 7, the rotating ring 280 is supported on the handle housing 110 and rotatable relative thereto by the rotating ring bearing 290. The rotating ring bearing 290 includes an outer ring 292 coupled (e.g., fixed) to the rotating ring 280 and an inner ring 294 coupled (e.g., fixed) to the first and second housing halves 112a, 112b of the handle housing 110. The outer ring 292 of the rotating ring bearing 290 is rotatable relative to the inner ring 294 such that the rotating ring 280 is rotatable with respect to the proximal and distal housings 112, 114 of the handle assembly 110.

In a method of use, the endoscope 100 is introduced into a surgical site and operated within methods known by those skilled in the art. A user changes the field of view by rotating the endoscope 100 by, for example, applying torque to the handle assembly 110. Rotation of the endoscope 100 results in a corresponding rotation of the imaging device 150 and thus, the imaging device 150 may be rotated within the tip 130 to change the orientation of the images of the surgical site that are displayed for the user. The user applies torque to the rotating ring 280 so that the rotating ring 280 rotates relative to the handle assembly 110. As the rotating ring 280 rotates, the magnet(s) 282 of the rotating ring 280 rotates and attracts the magnet(s) 252 of the magnet holder 250 so that the magnet holder 250 is pulled in the direction of rotation. Rotation of the magnet holder 250 drives rotation of the rotation shaft 240 which, in turn, rotates the lens plate 220 via the cross shaft 230. As the lens plate 220 is secured to the lens 152, the lens 152 is thus rotated relative to the tip 130.

While embodiments have been described, other embodiments are possible and it should be understood that various modifications may be made without departing from the spirit and scope of the disclosure. For example, the tip 130 of the endo scope 100 may be configured to rotate relative to the elongate shaft 120 such that rotation of the rotating ring 240 results in rotation of the tip 130 and thus, rotation of the imaging device 150 of the endoscope 100.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. An endoscope comprising:
a handle assembly;
an elongate shaft extending distally from the handle assembly along a central longitudinal axis;
a tip disposed at a distal end of the elongate shaft;
an imaging device disposed within the tip; and
a rotation assembly operably coupled to the imaging device and extending from the tip, through the elongate shaft, and into the handle assembly for rotating the imaging device within the tip.

2. The endoscope of claim 1, wherein the tip includes a distal surface extends along an axis that is angled with respect to the central longitudinal axis of the elongate shaft.

3. The endoscope of claim 2, wherein the imaging device is disposed within a cavity defined in the tip, and a transparent imaging cover is disposed over the imaging device and positioned within an imaging window defined in the distal surface of the tip.

4. The endoscope of claim 2, wherein the imaging device includes a lens extending through the tip along a longitudinal axis perpendicular to the axis defined by the distal surface of the tip.

5. The endoscope of claim 4, wherein the rotation assembly includes a lens bearing supporting the lens within the tip, the lens bearing including an outer ring coupled to the tip and an inner ring coupled to the lens, the inner ring rotatable relative to the outer ring such that the lens is rotatable within the tip.

6. The endoscope of claim 4, wherein the rotation assembly includes a lens plate secured to the lens of the imaging device, and a rotation shaft operably coupled to the lens plate, wherein rotation of the rotation shaft rotates the lens plate and the lens.

7. The endoscope of claim 6, wherein the rotation assembly includes a cross shaft interconnecting the lens plate and the rotation shaft such that the lens plate and the rotation shaft are pivotably coupled to one another about two axes defined by the cross shaft.

8. The endoscope of claim 6, wherein the rotation shaft extends from the lens plate proximally through the elongate shaft and into the handle assembly, the rotation shaft radially rotatable around the central longitudinal axis.

9. The endoscope of claim 8, wherein the rotation assembly includes a rotation shaft bearing supporting the rotation shaft in the handle assembly.

10. The endoscope of claim 6, wherein the rotation assembly includes a magnet base disposed within the handle assembly and radially rotatable therein around the central longitudinal axis, the magnet base including at least one magnet, the rotation shaft extending through the magnet base in fixed relation relative thereto.

11. The endoscope of claim 10, wherein the rotation assembly includes a rotating ring disposed on the handle assembly and rotatable relative to the handle assembly, the rotating ring including at least one magnet having a polarity opposite a polarity of the at least one magnet of the magnet base such that rotation of the rotating ring causes a corresponding rotation of the magnet base and the rotation shaft.

12. The endoscope of claim 11, wherein the at least one magnet of the magnet base is a plurality of magnets disposed radially around the magnet base in spaced relation relative to each other, and the at least one magnet of the rotating ring is a plurality of magnets disposed radially around the rotating ring in spaced relation relative to each other, wherein each magnet of the plurality of magnets of the magnet base is paired with one of the plurality of magnets of the rotating ring, the paired magnets having opposite polarities.

13. The endoscope of claim 12, wherein the plurality of magnets of the magnet base alternate in polarity around the magnet base, and the plurality of magnets of the rotating ring alternate in polarity around the rotating ring.

14. The endoscope of claim 11, wherein the handle assembly includes a proximal housing formed from first and second housing halves that cooperate to define a grip portion and a mount portion, the rotating ring supported on the mount portion of the proximal housing.

15. The endoscope of claim 14, wherein the rotation assembly includes a rotating ring bearing including an outer ring coupled to the rotating ring and an inner ring coupled to the mount portion of the proximal housing, the outer ring rotatable relative to the inner ring such that the rotating ring is rotatable relative to the proximal housing.

16. The endoscope of claim 14, wherein the handle assembly includes a distal housing, and the second housing half of the proximal housing extends distally beyond the first housing half and supports the distal housing thereon.

17. The endoscope of claim 16, wherein the distal housing defines a central opening therethrough in which the elongate shaft is supported and connected to the handle assembly.

18. A method of changing the field of view of an endoscope comprising:
rotating a rotation assembly of an endoscope, the rotation assembly operably coupled to an imaging device disposed within a tip of the endoscope to change the orientation of the imaging device relative to the tip, the rotation assembly extending from the tip, through an elongated shaft of the endoscope, and into a handle assembly of the endoscope.

19. The method of claim 18, wherein rotating the rotation assembly includes applying torque to a rotating ring of the rotation assembly, the rotating ring disposed on the handle assembly.

20. The method of claim 18, further comprising rotating the endoscope in its entirety to cause a corresponding rotation of the elongate shaft and the tip, the tip including a distal surface that extends along an axis that is angled with respect to a central longitudinal axis of the elongate shaft.
